(19) United States
(12) Patent Application Publication
(10) Pub. No.: US 2015/0290265 A1
(43) Pub. Date: Oct. 15, 2015

(54) COMPOSITION FOR TREATING METABOLIC DISORDERS

(71) Applicant: PIRAMAL ENTERPRISES LIMITED, Mumbai (IN)

(72) Inventors: Arvind SAKLANI, MUMBAI (IN); Nilesh MALPURE, Hadapsar, Pune (IN); Parikshit GAIKWAD, Mumbai (IN); Satish Namdeo SAWANT, Mumbai (IN); Tukaram Kisanrao MANE, Mumbai (IN); Somesh SHARMA, Los Altos, CA (US)

(21) Appl. No.: 14/391,762

(22) PCT Filed: Apr. 22, 2013
(86) PCT No.: PCT/IB2013/053155

§ 371 Date: Oct. 10, 2014

Related U.S. Application Data
(60) Provisional application No. 61/636,792, filed on Apr. 23, 2012.

(51) Int. Cl. A61K 36/185; A61K 31/7048

(52) U.S. Cl. CPC A61K 36/185; A61K 31/7048

(57) ABSTRACT

The present invention relates to a herbal composition comprising a therapeutically effective amount of an extract of the plant Terminalia elliptica as an active ingredient and optionally, a pharmaceutically acceptable carrier. The invention also relates to a process for the preparation of the extract. The invention also relates to a method for the treatment of metabolic disorders using the composition. The present invention also relates to a composition comprising a therapeutically effective amount of an extract of the plant Terminalia elliptica for use in combination with one or more further therapeutically active agent for the treatment of metabolic disorders.

### FIELD OF THE INVENTION

The present invention relates to a herbal composition comprising an extract of the plant, Terminalia elliptica as an active ingredient either alone, or with a pharmaceutically acceptable carrier. The composition of the present invention is useful for the treatment of metabolic disorders. The present invention also relates to a process for the preparation of the herbal composition.

### BACKGROUND OF THE INVENTION

Metabolic disorders are the disorders or defects that occur when the body is unable to properly metabolise carbohydrates, lipids, proteins, or nucleic acids. Most metabolic disorders are caused by genetic mutations that result in missing or dysfunctional enzymes that are needed for the cell to perform metabolic processes. Examples of metabolic disorders include obesity, excessive body fat, hyperlipidemia, hyperlipoproteinemia, hyperglycemia, hypercholesterolemia, hyperinsulinemia, insulin resistance, glucose intolerance and diabetes mellitus particularly type 2 diabetes. Considering the drawbacks associated with the existing drugs, there is a need to provide/develop new drugs for the treatment of metabolic disorders.

In order to select and develop new drug candidates for the treatment of metabolic disorders, two novel enzyme targets, Diacylglycerol Acyltransferase-1 (DGAT-1) and Stearoyl-CoA Desaturase-1 (SCD-1) can be utilised. These enzymes play a key role in the synthesis of triglyceride, the main form in which energy is stored in the body.

DGAT-1 is an endoplasmic membrane-bound enzyme that catalyses the biosynthesis of triglyceride at the final step of the process, converting diacylglycerol (DAG) and fatty acyl-coenzyme A (CoA) into triglyceride. The enzymatic activity is present in all cell types because of the necessity of producing triglyceride for cellular needs. DGAT-1 is highly expressed in the intestine and adipose with lower levels in the liver and muscle. Inhibition of DGAT-1 in each of these tissues (intestine, adipose, liver and muscle) would inhibit triacylglycerol synthesis and may reverse the pathophysiology of excessive lipid accumulation in human metabolic disease (Expert Opin. Ther. Patents, 17(11), 1331-1339, 2007).

Stearoyl-CoA Desaturase-1 (SCD-1), has been described as one of the major enzymes in the control of lipid metabolism and may represent a potential new therapeutic target. SCD-1 is a rate-limiting enzyme that catalyzes the biosynthesis of monounsaturated fatty acids from saturated fatty acids. The preferred substrates of SCD-1, stearate (C18:0) and palmitate (C16:0), are converted to oleate (C18:1) and palmitoyleate (C16:1) respectively. These monounsaturated fatty acids are considered as the major components of various lipids including triglycerides, cholesteryl esters, phospholipids and wax esters. Studies in experimental animals suggest that inhibiting or reducing the activity of these enzymes results in resistance to development of obesity, diabetes and associated complications (European Journal of Pharmacology, 618, 28-36, 2009), European Journal of Pharmacology, 650, 663-672, 2011).

In the modern era of medicine, herbal materials and plants continue to play an important role in drug discovery and development. The demand for plant-based medicines is ever growing since crude or processed products obtained from plants are believed to have fewer or no adverse effects as compared to the drugs that are synthetic small molecules.

“Terminalia” is a genus of large trees of the flowering plant family Combretaceae, comprising around hundred species distributed in tropical regions of the world. The most commonly known plants of Terminalia genus are Terminalia bellirica, Terminalia catappa, Terminalia paniculata, Terminalia citrina, Terminalia phellocarpa, Terminalia copelandii, Terminalia brassi, Terminalia ivorensis, Terminalia superba, Terminalia arjuna, Terminalia elliptica and Terminalia chebula. Trees of this genus are known especially as a source of secondary metabolites, e.g. cyclic triterpenes and their derivatives, flavonoids, tannins, and other aromatics. The extract obtained from the plant, Terminalia bellirica, particularly that obtained from the fruits without seeds, has been shown to have α-glucosidase inhibition effect (Japanese Application Publication No. JP 2006-188486). It is also reported in JP 2006-188486 that fruits of the plant, Terminalia chebula showed a weak α-glucosidase inhibition effect.

Terminalia elliptica is a species of Terminalia, native to southern and southeast Asia in India, Nepal, Bangladesh, Myanmar, Thailand, Laos, Cambodia, and Vietnam. The synonyms of Terminalia elliptica include Terminalia tomentosa, Terminalia crenulata, Terminalia alata, Terminalia coriaceana and Pentaptera crenulata.

Terminalia elliptica is a large, deciduous tree growing up to thirty meter tall, with trunk of a diameter of one meter. The bark of Terminalia elliptica is rough and is deeply cracked. The outer surface is pale brown to dark brown in colour and the inner surface is dark brown to black in colour, smooth and longitudinally striated. The bark is bitter and styptic and is useful in treating ulcers, fractures, haemorrhages and bronchitis. The bark has both diuretic and cardiotonic properties. A decoction of bark is taken internally in atonic diarrhoea and locally as an application to weak indolent ulcers (Glossary of Indian Medicinal Plants. CSIR, New Dehli, ISBN: 8172361262, 1956). Sushruta recommends the ashes of the plant in the treatment of snake bite (Indian Medicinal Plants, Dehradun, India. Vol. II, pp. 1028, 1984).

The leaves of Terminalia elliptica are used as food by Antheraea paphia (silkworms) which produce the tassar silk. The flowers of Terminalia elliptica are pale yellow, hermaphrodite and present in spikes or terminal panicles. The flowering season is from March to June.

It has been indicated herein above that considering the growing prevalence of metabolic disorders such as type 2 diabetes and obesity, there exists a continuing need for new compositions and methods for the effective treatment of the metabolic disorders. In fact, efforts of the inventors of the present invention directed to find a solution to these problems have resulted in a herbal composition comprising an extract of the plant, Terminalia elliptica, having dual DGAT-1 and SCD-1 inhibitory activity, and hence is useful for the treatment of metabolic disorder.

### SUMMARY OF THE INVENTION

According to one aspect of the present invention, there is provided a composition comprising a therapeutically effective amount of an extract of the plant, Terminalia elliptica, as an active ingredient and optionally, at least one pharmaceutically acceptable carrier, for use in the treatment of a metabolic disorder.

According to another aspect of the present invention, there is provided a composition comprising a therapeutically effective amount of an extract of the plant, Terminalia elliptica, for use in combination with a further therapeutically active agent, for the treatment of a metabolic disorder.

In another further aspect, the present invention is directed to a method for the treatment of a metabolic disorder in a subject comprising administering to the subject, a composition comprising a therapeutically effective amount of an extract of the plant, Terminalia elliptica, as an active ingredient and optionally at least one pharmaceutically acceptable carrier.

In another further aspect, the present invention is directed to a method for the treatment of a metabolic disorder in a subject comprising administering to the subject, a composition comprising a therapeutically effective amount of an extract of the plant, Terminalia elliptica, as an active ingredient and optionally, at least one pharmaceutically acceptable carrier, wherein said method comprises administering the composition in combination with a further therapeutically active agent.

According to another aspect of the present invention, there is provided a process for the preparation of the composition, comprising a therapeutically effective amount of the extract of the plant, Terminalia elliptica and at least one pharmaceutically acceptable carrier.

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the description herein, may utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs.

The term “metabolic disorder” refers to the disorders or defects that occur when the body is unable to properly metabolise carbohydrates, lipids, proteins, or nucleic acids. Accordingly, in the context of the present invention all the disorders relating to abnormality of metabolism are encompassed in the term “metabolic disorders”. The term metabolic disorders include, but not limited to, insulin resistance, hyperglycemia, diabetes mellitus, obesity, glucose intolerance, hypercholesterolemia, dyslipidemia, hyperinsulinemia, atherosclerotic disease, polycystic ovary syndrome, coronary artery disease, metabolic syndrome, hypertension, or a related disorder associated with abnormal plasma lipoprotein, triglycerides or a disorder related to glucose levels such as pancreatic beta cell regeneration.

The term “treating”, “treat” or “treatment” as used herein includes preventive (prophylactic) and palliative treatment.

The term “pharmaceutically acceptable” as used herein means the carrier, diluent, excipients, and/or salt used in the composition must be compatible with the other ingredients of the formulation, and not deleterious to the recipient thereof.

The terms “herbal composition” or “composition” are used interchangeably and may refer to a composition comprising a therapeutically effective amount of the extract of the plant Terminalia elliptica either alone or with at least one pharmaceutically acceptable carrier or excipient. The term “either alone” may further indicate that the composition contains only the extract of the plant Terminalia elliptica without any pharmaceutically acceptable carrier added therein. It should be noted that the term “composition” should be construed in a broad sense and includes any composition which is intended for the purpose of achieving a therapeutic effect whether sold as a pharmaceutical product, for example carrying a label as to the intended indication, whether sold over the counter, or whether sold as a phytopharmaceutical.

The term “Terminalia elliptica” as used herein includes all its synonyms such as Terminalia tomentosa, Terminalia crenulata, Terminalia alata, Terminalia coriaceana and Pentaptera crenulata.

The term “pharmaceutically acceptable carrier” as used herein means a non-toxic, inert solid, semi-solid, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose and cellulose acetate; malt; gelatin; as well as other non-toxic compatible lubricants such as sodium lauryl sulfate and magnesium stearate, as well as coloring agents, releasing agents, coating agents, sweetening, flavoring and perfuming agents; preservatives and antioxidants can also be used in the composition, according to the judgment of the formulator.

The term “therapeutically effective amount” as used herein means an amount of the extract (the “Terminalia elliptica” extract) or the composition containing the extract, which is sufficient to significantly induce a positive modification in the condition to be regulated or treated, but low enough to avoid side effects, if any (at a reasonable benefit/risk ratio), within the scope of sound medical judgment. The therapeutically effective amount of the extract or composition will vary with the particular condition being treated e.g. diabetes mellitus or obesity, the age and physical condition of the end user, the severity of the condition being treated/prevented, the duration of the treatment, the nature of concurrent therapy, the particular pharmaceutically acceptable carrier utilized, and like factors. As used herein, all percentages are by weight unless otherwise specified.

It should be noted that, as used in the specification and the appended claims, the singular forms “a,” “an,” and “the” include plural referents unless the content clearly dictates otherwise.

The term “Terminalia elliptica extract” or “the extract of Terminalia elliptica” as used herein means a blend of compounds present in any part of the plant Terminalia elliptica. Such compounds can be extracted from any part of the plant, such as the bark, twig, stem, wood, leaves and fruit of the plant, using extraction procedures well known in the art e.g., by carrying out the extraction procedure using organic solvents such as lower alcohols e.g. methanol or ethanol, alkyl esters such as ethyl acetate, alkyl ethers such as diethyl ether, alkyl ketones such as acetone, chloroform, petroleum ether, hexane and/or an aqueous solvent such as water. The plant material can also be extracted by using a mixture of solvents in a suitable ratio for example, hexane-ethyl acetate (1:1), chloroform-methanol (1:1) or methanol-water (3:1).

The term “subject” as used herein refers to an animal, particularly a mammal, and more particularly, a human. The term “mammal” used herein refers to warm-blooded vertebrate animals of the class Mammalian, including humans, characterized by a covering of hair on the skin and, in the female, milk-producing mammary glands for nourishing the young. The term mammal includes animals such as cat, dog, rabbit, bear, fox, wolf, monkey, deer, mouse, pig and the human.

In an embodiment, the process for the preparation of “Terminalia elliptica extract” involves use of an alcohol e.g. methanol as the solvent.

For example, the extract can be obtained by extraction of any part of the plant, Terminalia elliptica e.g. the bark.

In an embodiment, the extract is obtained from the pulverized bark of the plant, Terminalia elliptica using methanol as the solvent.

In an embodiment, the extract is obtained from the pulverized bark of the plant, Terminalia elliptica using a mixture of solvents in suitable ratio.

In an embodiment, the pulverized bark of the plant Terminalia elliptica can be extracted using methanol-water mixture in different ratios, e.g. methanol-water (9:1) mixture, methanol-water (3:1) mixture or methanol-water (1:1) mixture can be used for extraction.

The process for preparation of the extract of the plant Terminalia elliptica can be easily scaled up for large-scale preparation by following a conventional approach.

Terminalia elliptica extract can be standardized using conventional techniques such as high performance liquid chromatography (HPLC) or high performance thin-layer chromatography (HPTLC). The term “standardized extract” refers to an extract which is standardized by identifying characteristic bioactive ingredient(s) or bioactive marker (s) present in the extract.

The term “active ingredient” as used herein refers to Terminalia elliptica extract containing a blend of compounds or the extract of the plant, Terminalia elliptica containing one or more bioactive compounds (bioactive markers).

Bioactive markers or bioactive ingredients can be identified using various techniques such as high performance thin-layer chromatography (HPTLC) or high performance liquid chromatography (HPLC). Bioactive markers can be isolated from the extract of the plant Terminalia elliptica by bioactivity guided column chromatographic purification and preparative high performance liquid chromatography (HPLC). Bioactive markers can be characterized by analysis of the spectral data.

The term “bioactive marker” is used herein to define a characteristic (or a phytochemical profile) of an active compound which is correlated with an acceptable degree of pharmaceutical activity. “Bioactive marker”, which is the active compound, can be isolated from the extract obtained from the plant, Terminalia elliptica by bioactivity guided

The isolated compounds (bioactive markers) may be characterized by analysis of the spectral data such as mass spectrum (MS), infra red (IR) and nuclear magnetic resonance (NMR) spectroscopic data.

In an embodiment, the bioactive marker isolated from the plant Terminalia elliptica was characterized as Ellagic acid, 4-O-alpha-L-rhamnopyranoside (herein after referred to as “the compound 1”).

The biological activity determination of the extracts can be carried out using various well-known biological in vitro and in vivo assays. For example, preliminary in vitro activity determination of the extracts can be carried out using assays such as Diacylglycerol Acyltransferase-1 (DGAT-1) assay, Stearoyl-CoA Desaturase-1 (SCD-1) assay or triglyceride synthesis assay. The in vivo activity can be determined by using assays such as the high fat diet (HFD) induced obesity model.

In an embodiment, the invention provides a herbal composition comprising a therapeutically effective amount of an extract of the plant, Terminalia elliptica and optionally at least one pharmaceutically acceptable carrier.

In another embodiment, the invention relates to a herbal composition comprising standardized extract of the plant Terminalia elliptica and optionally, at least a pharmaceutically acceptable carrier.

The term “standardized extract” as used herein refers to an extract of a plant e.g. “Terminalia elliptica” that has been processed so that it contains in specified amount a compound as a bioactive marker. In the context of the present invention, the term standardized extract refers to the extract of the plant Terminalia elliptica containing specified amount of the compound 1, as the bioactive marker. The specified amount of the compound 1 present in the standardized extract may vary from 0.01% to 10% or from 0.05% to 5% or 0.15% to 2%.

In an embodiment, the standardized extract of the plant Terminalia elliptica contains 0.01% to 10.0% of the compound 1, as the bioactive marker.

In another embodiment, the standardized extract of the plant Terminalia elliptica contains 0.05% to 5.0% of the compound 1, as the bioactive marker.

In another embodiment, the standardized extract of the plant Terminalia elliptica contains 0.15% to 2.0% of the compound 1, as the bioactive marker.

In another embodiment, the invention relates to a herbal composition comprising standardized extract of the plant Terminalia elliptica containing 0.01% to 10.0% of the compound 1 (Ellagic acid, 4-O-alpha-L-rhamnopyranoside) as the bioactive marker, and optionally, at least a pharmaceutically acceptable carrier.

In an embodiment, the invention provides a herbal composition comprising a therapeutically effective amount of an extract of the bark of the plant Terminalia elliptica and optionally at least one pharmaceutically acceptable carrier.

In an embodiment, the invention provides a herbal composition comprising a therapeutically effective amount of an extract of the stem of the plant Terminalia elliptica and optionally at least one pharmaceutically acceptable carrier.

The herbal composition of the present invention comprises 5%-100% of the extract of the plant Terminalia elliptica.

In an embodiment, the invention provides a herbal composition comprising 45%-75% of the extract of the plant Terminalia elliptica.

The herbal composition of the present invention comprises 5%-100% of the extract, obtained from the plant Terminalia elliptica containing at least 0.01% to 10.0% of the compound 1 as the bioactive marker.

In an embodiment, the invention provides a herbal composition comprising 45%-75% of the extract of the plant Terminalia elliptica containing at least 0.05% to 5.0% of the compound 1 as the bioactive marker.

In an embodiment, the invention provides a herbal composition comprising 45%-75% of the extract of the plant Terminalia elliptica containing at least 0.15% to 2.0% of the compound 1 as the bioactive marker.

In an embodiment, the invention provides use of the composition comprising a therapeutically effective amount of the extract of the plant Terminalia elliptica, for the manufacture of a medicament for the treatment of metabolic disorders.

In an embodiment, the extract of the plant Terminalia elliptica contained in the composition is the standardized extract.

The Terminalia elliptica extract is mixed with pharmaceutically acceptable carriers and formulated into therapeutic dosage forms.

The compositions comprising a therapeutically effective amount of the extract of the plant Terminalia elliptica can be administered orally, for example in the form of pills, tablets, coated tablets, capsules, powders, granules, elixirs or syrup.

The oral compositions containing 5-100% by weight of the Terminalia elliptica extract can be prepared by thoroughly mixing the extract with pharmaceutically acceptable carrier/s, by using conventional methods.

The compositions of the present invention can be used for transdermal administration.

In an embodiment, the said compositions are provided for the treatment of a metabolic disorder.

In an embodiment, the metabolic disorder is selected from insulin resistance, hyperglycemia, diabetes mellitus, obesity, glucose intolerance, hypercholesterolemia, dyslipidemia, hyperinsulinemia, atherosclerotic disease, polycystic ovary syndrome, coronary artery disease, metabolic syndrome, hypertension, disorders associated with abnormal plasma lipoprotein, triglycerides or a disorder related to pancreatic beta cell regeneration.

In another embodiment, the metabolic disorder is selected from: insulin resistance, diabetes mellitus, hyperglycemia, metabolic syndrome, glucose intolerance, obesity, dyslipidemia, disorders associated with abnormal plasma lipoprotein, triglycerides or a disorder related to pancreatic beta cell regeneration.

In an embodiment the said composition is provided for the treatment of diabetes mellitus.

The term “diabetes mellitus” or “diabetes” refers to a chronic disease or condition, which occurs when the pancreas does not produce enough insulin, or when the body cannot effectively use the insulin it produces. This leads to an increased concentration of glucose in the blood (hyperglycaemia). Two major forms of diabetes are type 1 diabetes (Insulin-dependent diabetes mellitus) and type 2 diabetes (Non-insulin dependent diabetes mellitus (NIDDM)). Type 1 diabetes is an autoimmune condition in which the insulin-producing β-cells of the pancreas are destroyed which generally results in an absolute deficiency of insulin, the hormone that regulates glucose utilization. Type 2 diabetes often occurs in the face of normal, or even elevated levels of insulin and can result from the inability of tissues to respond appropriately to insulin. Other categories of diabetes include gestational diabetes (a state of hyperglycemia which develops during pregnancy) and “other” rarer causes (genetic syndromes, acquired processes such as pancreatitis, diseases such as cystic fibrosis, exposure to certain drugs, viruses, and unknown causes).

In an embodiment of the invention, the term diabetes or diabetes mellitus refers to type 2 diabetes (Non-insulin dependent diabetes mellitus(NIDDM)).

In an embodiment the said composition is provided for the treatment of obesity.

In an embodiment the said composition is provided for the treatment of dyslipidemia.

In an embodiment the said compositions are provided for the treatment of metabolic disorders related to disorders associated with abnormal plasma lipoprotein, triglycerides.

In an embodiment the said compositions are provided for the treatment of metabolic disorders related to glucose levels such as pancreatic beta cell regeneration.

In yet another embodiment, the present invention relates to a composition comprising a therapeutically effective amount of an extract of the plant Terminalia elliptica, for use in combination with at least one further therapeutically active agent for use in the treatment of a metabolic disorder.

In yet another embodiment, the present invention relates to a composition comprising a therapeutically effective amount of the extract of the plant Terminalia elliptica and optionally, at least a pharmaceutically acceptable carrier, for use in combination with at least one further therapeutically active agent, for use in the treatment of a metabolic disorder.

The therapeutically active agent that may be combined with the composition of the present invention may be selected from the extract of the plants selected from Calophyllum inophyllum, Pterospermum acerifolium, Tinospora cardifolia, Capsicum annum, Galega officinalis or Allium sativum.

The therapeutically active agent that may be combined with the composition of the present invention may also be selected from the known therapeutic agents such as orlistat, pioglitazone, rosiglitazone, glibenclamide, glipizide, glimeperide, repaglinide, nateglinide, or metformin.

Moreover, the composition of the present invention may be combined with one or more of the further therapeutic agents which may be selected from the extract of the plants selected from Calophyllum inophyllum, Pterospermum acerifolium, Tinospora cardifolia, Capsicum annum, Galega officinalis or Allium sativum and the known drugs selected from orlistat, pioglitazone, rosiglitazone, glibenclamide, glipizide, glimeperide, repaglinide, nateglinide, or metformin.

The present invention is also related to a method of treating a metabolic disorder comprising the administration of the composition comprising a therapeutically effective amount of the extract of the plant Terminalia elliptica and optionally, at least a pharmaceutically acceptable carrier, selectively by oral route.

The herbal composition of the present invention may be formulated for oral administration by compounding the active ingredient i.e. the extract of the plant Terminalia elliptica which may be a standardized extract with the usual non-toxic pharmaceutically acceptable carrier/s for powders, pills, tablets, coated tablets, pellets, granules, capsules, solutions, emulsions, suspensions, elixirs, syrup, and any other form suitable for use. Formulations of the present invention encompass those which include talc, water, glucose, lactose, sucrose, gum acacia, gelatin, mannitol, starch paste, magnesium trisilicate, corn starch, keratin, colloidal silica, potato starch, urea, and cellulose and its derivatives such as sodium carboxymethyl cellulose, ethyl cellulose and cellulose acetate; malt; gelatin; as well as other non-toxic compatible lubricants such as sodium lauryl sulfate and magnesium stearate, releasing agents, coating agents and other excipients suitable for use in manufacturing preparations, in solid, semisolid or liquid form and in addition auxiliary, stabilizing, thickening and coloring agents may be used. For preparing solid compositions such as tablets or capsules, the extract is mixed with a pharmaceutical carrier (e.g., conventional tableting ingredients such as corn starch, lactose, sucrose, sorbitol, talc, stearic acid, magnesium stearate, dicalcium phosphate or gums) and other pharmaceutical diluents (e.g., water) to form a solid composition. This solid composition is then subdivided into unit dosage forms containing an effective amount of the composition of the present invention. The tablets or pills containing the extract can be coated or otherwise compounded to provide a dosage form affording the advantage of prolonged action.

The liquid forms, in which the extract of the plant Terminalia elliptica which may be a standardized extract may be incorporated for oral or parenteral administration, include aqueous solution, suitably flavored syrups, aqueous or oil suspensions, and flavored emulsions with edible oils as well as elixirs and similar pharmaceutical vehicles. Suitable dispersing or suspending agents for aqueous suspensions include synthetic natural gums, such as tragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

The selected dosage level will depend upon a variety of factors including the activity of the particular extract of the present invention employed, the route of administration, the time of administration, the rate of excretion of the particular composition being employed, the duration of the treatment, used in combination with the other extracts, the age, sex, weight, condition, general health and prior medical history of the patient being treated, and like factors well known in the medical arts. In general, however, doses employed for human treatment will typically be in the range of 1-5000 mg per day. In any case the required dose may be increased or decreased depending on the severity of the disease and the other parameters by the medical practitioner. For example, the doses in which the composition can be used may be 1-1500 mg/day or 5-1000 mg/day or 10-1000 mg/day or 5-500 mg/day or any other suitable dose. The desired dose may conveniently be presented in a single dose or as divided doses administered at appropriate intervals, for example as two, three, four or more sub-doses per day.

The present invention will be more readily understood by referring to the following examples which are given to illustrate the invention but do not limit its scope.

### EXAMPLES

The following terms/abbreviations are employed in the examples:

| | |
|---|---|
| L: Litre | KCl: Potassium chloride |
| mL: Millilitre | NaOH: Sodium hydroxide |
| μL: Microlitre | MgCl₂: Magnesium chloride |
| g: Gram | KH₂PO₄: Potassium dihydrogen |
| | phosphate |
| mg: Milligram | K₂HPO₄: Dipotassium hydrogen |
| | phosphate |
| μg: Microgram | DMSO: Dimethyl sulfoxide |
| M: Molar | cpm: Counts per minute |
| mM: Millimolar | rpm: Revolutions per minute |
| μM: Micromolar | dpm: Disintegrations per minute |
| nM: Nanomolar | pfu: Plaque forming units |
| mm: millimeter | AESSM: Alkaline Ethanol Stop |
| | Solution Mix |
| cm: centimeter | BSA: Bovine Serum Albumin |
| μ: Micron | DAB: DGAT Assay Buffer |
| w/v: Weight by volume | EDTA: Ethylene Diamine |
| | Tetraacetic Acid |
| μg/mL: Microgram per millilitre | FBS: Fetal Bovine Serum |
| ng/μL: Nanogram per microlitre | PBS: Phosphate Buffered Saline |
| mg/kg: Milligram per kilogram | ORF: Open Reading Frame |
| h: Hours | RZPD: German Resource Center |
| min: Minutes | MOI: Multiplicity of infection |
| RT: Room Temperature | EMEM: Eagle's Minimum |
| (25 ± 5° C.) | Essential Medium |

β-NADH: β-Nicotinamide Adenine Dinucleotide
Tris-HCl buffer: Tris(hydroxymethyl)aminomethane —HCl buffer
NaH₂PO₄•2H₂O: Sodium dihydrogen phosphate dihydrate
Sf9 cells: Clonal isolate, derived from Spodoptera frugiperda
HepG2 Cells: Human liver hepatocellular carcinoma cell line
HPLC: High Performance Liquid Chromatography

### Extractions of the Plant

Bark of the plant Terminalia elliptica was procured from the IVYS Agro, Pune, India.

A microscopic and macroscopic study for authentication was carried out for the bark of the plant Terminalia elliptica, and a specimen has been retained in Botany Department, Piramal Healthcare Limited, Goregaon, Mumbai, India.

The bark of the plant was chopped into small pieces and was dried with the help of dehumidifier. The completely dried material was then coarsely ground using a pulveriser.

### Example 1

Dried pulverized bark of Terminalia elliptica (200 g) was extracted using methanol (2 L) by stirring at 45° C. for 3 h. This extraction process was repeated twice with methanol (1.6 L). The extracts were combined and concentrated to dryness. Yield: 41.18 g (20.59%).

Extract so obtained in Example 1 is referred to as “Extract of Example 1”.

The Extract of Example 1 was found to contain 0.71% of the bioactive marker (the compound 1; estimation by analytical HPLC method described in Example 5).

The Extract of Example 1 was stored in polypropylene vial in cold room at 4° C. to 8° C.

### Example 2

Dried pulverized bark of Terminalia elliptica (100 g) was extracted using methanol:water (9:1) (1 L) by stirring at 45° C. for 3 h. This extraction process was repeated twice with methanol:water (9:1) (700 mL). The extracts were combined and concentrated. The concentrated material was lyophilized using freeze-dryer (Edwards). Yield: 5.2 g (5.2%).

Extract so obtained in Example 2 is referred to as “Extract of Example 2”.

The Extract of Example 2 was found to contain 0.89% of the bioactive marker (the compound 1; estimation by analytical HPLC method described in Example 5).

The Extract of Example 2 was stored in polypropylene vial in cold room at 4° C. to 8° C.

### Example 3

Dried pulverized bark of Terminalia elliptica (50 g) was extracted using methanol:water (3:1) (500 mL) by stirring at 40° C.±5° C. by for 3 h. This extraction process was repeated twice with methanol:water (3:1) (400 mL). The extracts were combined and concentrated. The concentrated material was lyophilized using freeze-dryer (Edwards). Yield: 7.6 g (15.12%). The extract was stored in polypropylene vial in cold room at 4° C. to 8° C.

The Extract of Example 3 was found to contain 0.49% of the bioactive marker (the compound 1; estimation by analytical HPLC method described in Example 5).

### Example 4

Dried pulverized bark of Terminalia elliptica (50 g) was extracted using distilled water (500 mL) by stirring at 40° C.±5° C. by for 3 h. This extraction process was repeated with distilled water (400 mL). The extracts were combined and concentrated. The concentrated material was lyophilized using freeze-dryer (Edwards). Yield: 6.3 g (12.6%). The extract was stored in polypropylene vial in cold room at 4° C. to 8° C.

The Extract of Example 4 was found to contain 0.61% of the bioactive marker (the compound 1; estimation by analytical HPLC method as described in Example 5).

### Example 5

### Isolation of the Bioactive Marker

### The Compound 1

The Extract of Example 1 was analysed by analytical HPLC (conditions as given below):

Column: Unisphere aqua C18, 150 mm×4.6 mm, 3μ

Gradient:

| Time | Mobile phase A (%) | Mobile phase B (%) |
|---|---|---|
| (min) | (0.1% Trifluoroacetic acid) | (Acetonitrile) |
| 0 | 90 | 10 |
| 15 | 60 | 40 |
| 25 | 10 | 90 |
| 26 | 90 | 10 |
| 30 | 90 | 10 |

Run time: 30 min; Concentration: 10 mg/mL in Methanol

Injection volume: 10 μL; Flow rate: 1 mL/min; Detection: UV 254 nm

Peak at retention time of 9.5 min was a major peak and was identified as bioactive marker (the compound 1). This component was isolated and purified as described below.

To the Extract of Example 1 (100 g) water (8 L) and polyamide (300 g) was added. The mixture was stirred at 60° C. for 3 h and filtered, washed with water (2 L). To the residue obtained, methanol (8 L) was added and stirred for 16 h at RT, filtered. To the residue obtained, methanol (8 L) was added and stirred for 8 h at RT, filtered. Methanol extract filtrates were pooled and concentrated to obtain enriched extract (10 g).

Above extract enriched with bioactive marker (the compound 1; 5 g) was subjected to purification in lots (1.25 g) each using C18 flash chromatography (conditions as given below).

Column: Redisep C18, 43 g, 14 cm×2 cm

Gradient:

| Time | Mobile phase A (%) | Mobile phase B (%) |
|---|---|---|
| (min) | (0.1% Trifluoroacetic acid) | (Acetonitrile) |
| 0 | 90 | 10 |
| 15 | 60 | 40 |
| 30-35 | 20 | 80 |
| 36 | 90 | 10 |
| 41 | 90 | 10 |

Sample loading: 1.25 g dry charged using 4 g C18 material

Flow: 25 mL/min; Detection: UV 254 nm

Fractions were monitored by analytical HPLC. It was found that the fractions contained significant amount of the bioactive marker (the compound 1), on standing overnight (˜16 h) yielded crystalline solid. The fractions containing crystals were pooled, filtered and dried to obtain the bioactive marker (the compound 1; 113 mg).

Spectroscopic data of the bioactive marker: IR (KBr): 3379, 1728, 1621, 1501, 1441, 1339, 1188, 1130, 1048, 974, 918, 753 cm⁻¹; ¹HNMR (500 MHz, DMSO-d₆): δ 11.05 (s, 1H), 10.88 (br s, 1H), 10.72 (br s, 1H), 7.75 (s, 1H), 7.49 (s, 1H), 5.47 (s, 1H), 5.11 (br s, 1H), 4.94 (br s, 1H), 4.72 (br s, 1H), 4.00 (br s, 1H), 3.86 (br d, 1H, J=8.65), 3.55 (m, 1H), 3.31 (br s, 1H) and 1.15 (d, 3H J=6.2); ¹³CNMR (75 MHz, DMSO-d₆): δ 159.56, 159.41, 149.14, 146.82, 141.57, 140.04, 137.19, 136.85, 114.96, 112.25, 112.01, 110.85, 108.68, 108.02, 100.65, 72.23, 70.53, 70.42, 70.34 and 18.35; MS: m/z (ESI) 446.7 (M-).

On the basis of MS, IR and NMR spectroscopic data the bioactive marker was identified as Ellagic acid, 4-O-alpha-L-rhamnopyranoside (the compound 1). Further, the structure was confirmed by comparing the obtained spectroscopic data with the reported literature data (J. Nat. Products, 61, 901-906, 1998).

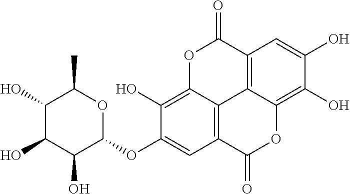 [SMILES: C[C@H]1O[C@H](Oc2cc3c(=O)oc4c(O)c(O)cc5c(=O)oc(c2O)c3c45)[C@@H](O)[C@@H](O)[C@@H]1O]

Bioactive marker ((the compound 1 or Ellagic acid, 4-O-alpha-L-rhamnopyranoside) was tested for in vitro biological activity, testing and the results are given in Example 6 and Example 7.

### Pharmacological Assays

The efficacy of the extract of the plant, Terminalia elliptica in inhibiting the activity of DGAT-1 and SCD-1 enzymes was determined by different pharmacological assays, well known in the art and are described below.

### In Vitro Assay

### Example 6

### hDGAT-1 Assay

The DGAT-1 assay was designed using human DGAT-1 enzyme over expressed in Sf9 cell-line as described in the reference, European Journal of Pharmacology, 650, 663-672, 2011, the disclosure of which is incorporated by reference for the teaching of the assay.

Cloning and Expression of Human DGAT-1 (hDGAT-1) Clone

hDGAT-1 ORF expression clone (RZPD0839C09146 in pDEST vector) was obtained from RZPD, Germany. hDGAT-1 gene (NM_{—}012079) was cloned into pDEST8 vector under strong polyhedron promoter of the Autographa californica nuclear polyhedrosis virus (AcNPV) with ampicillin resistance marker. The recombinant plasmid was introduced into DH10BAC competent cells (Invitrogen, US) by transformation which contains baculovirus shuttle vector (bacmid), and the resultant cells were streaked on to Luria broth (LB) agar plate containing ampicillin (100 μg/mL), kanamycin (50 μg/mL) and of gentamycin (10 μg/mL) according to the Bac-to-Bac baculovirus Expression System (Invitrogen, US). The white colonies were picked and restreaked on to LB agar plates having above antibiotics and incubated overnight at 37° C. On the following day isolated white colonies with recombinant bacmid containing hDGAT-1 gene were inoculated into 10 mL of Luria broth with antibiotics (ampicillin (100 μg/mL), kanamycin (50 μg/mL) and gentamycin (10 μg/mL)) and incubated overnight with 200 rpm at 37° C. in an orbital shaker (New Brunswick). 10 mL of Luria broth was taken and recombinant bacmid DNA (with hDGAT-1 gene) was prepared using the Qiagen mini prep kit and was quantified using nanodrop. The concentration of the bacmid DNA containing hDGAT-1 gene was approximately 97 ng/μL.

### Transfection and Virus Amplification Using Sf9 Cells

1-3 μg of hDGAT-1 bacmid DNA was transfected into Sf9 cells using Cellfectin (Invitrogen, US) according to manufacturer's specifications in 6-well tissue culture plates. Transfected Sf9 cells were incubated at 27° C. for 5 h in incomplete Grace's insect media (Gibco®) without fetal bovine serum and antibiotic-antimycotic (100 units/mL), penicillin, (100 μg/mL), streptomycin sulphate, (0.25 μg/mL) and amphotericin B. After completion of incubation media was replaced by growth media (Grace's insect media; (Gibco®) containing 10% fetal bovine serum (Hyclone) and antibiotic-antimycotic (100 units/mL), penicillin (100 μg/mL), streptomycin sulphate (0.25 μg/mL) and amphotericin B) and the cells were further incubated for 120 h at 27° C. in an incubator.

During this incubation, viral particles formed within the insect cells and were secreted. The supernatant containing the virus was collected at the end of 120 h by centrifuging at 1500×g for 5 min using Biofuge statos centrifuge (Heraeus 400), and was filtered through 0.22 μm filter (Millipore). It was stored as P1 recombinant baculovirus at 4° C. The cont >10⁵ pfu (plaque forming units)/mL were determined by the plaque assay conducted as per manufacturer's protocol (Invitrogen kit).

P1 recombinant baculovirus was further amplified at a MOI (multiplicity of infection) of 0.05-0.1, to generate P2 recombinant baculo virus in T-25 flask (Nunc) containing 5×10⁶ Sf9 cells in 5 mL complete Grace's insect media for 120 h followed by centrifugation at 1500×g for 5 min, filtration through 0.22 μm filter (Millipore), and storage at 4° C. as P2/(>106 pfu/mL) recombinant baculovirus. Similarly P3 and P4 recombinant baculovirus was further amplified, by reinfection at a MOI of 0.05-0.1, to generate P3 and P4 recombinant baculovirus respectively and were stored at 4° C. until further use. Viral titer for the P4 recombinant baculovirus was determined and it was found to be 1×10 pfu/mL. The P4 (>10⁸ pfu/mL) recombinant baculo virus was finally used to infect sf9 cells at a MOI of 5-10.

### Microsome Preparation

Sf9 cells (2×10⁶ Cells/mL) grown in a 500 mL spinner flask containing 250 mL of Grace's insect cell media (Gibco) with antibiotic-antimycotic (Gibco®) and were infected with hDGAT-1 recombinant baculovirus (25 mL) at an MOI of 5. The infected cells were maintained for 48 h at 28° C. and the cell pellet was collected by centrifuging the media at 1000×g at room temperature. The pellet was washed with PBS (pH 7.4) to eliminate residual media.

Cells were then disrupted by suspending the pellet in 15 mL of microsome preparation buffer containing IX amount of protease cocktail tablet (Roche) and in house prepared protease inhibitor mixture by passing the lysate through a 27G needle followed by mild sonication at 4° C. The cell debris was separated and the post nuclear supernatant (PNS), the lysate was centrifuged at 1000×g for 10 min at 4° C. using Biofuge statos centrifuge (Heraeus 400). The PNS obtained was then centrifuged at 15000×g for 30 min at 4° C. using the Biofuge statos centrifuge (Heraeus) to separate the post mitochondrial supernatant (PMS). Finally, ultracentrifugation was done at 100,000×g for 1 h at 4° C. using BeckmaTi-rotor to obtain microsomal pellet. To increase purity, the pellet was washed two times in microsomal preparation buffer containing in house preparation of a protease inhibitor mixture (Aprotinin (0.8 μM), pepstatin A (10 μM) and leupeptin (20 μM)-Sigma).

Finally microsomal pellet was suspended in 1.5 mL of the microsome preparation buffer and protein concentration was determined by Bradford method.

The microsomes were stored as aliquots of 100 μL each at −70° C. for in vitro assay.

### Preparation of Buffers and Reagents

### Stock Solutions

hDGAT-1 Assay Buffer Stock:

Assay buffer of pH 7.4 was prepared by dissolving 0.25 M sucrose (Sigma) and 1 mM EDTA (Sigma) in 150 mM tris HCl (Sigma).

Stop Solution:

For making 10 mL of Stop solution, 7.84 mL of isopropanol (Qualigens) and 1.96 mL of n-heptane (Qualigens) were added in 0.2 mL de-ionized water.

A.E.S.S.M (Alkaline Ethanol Stop Solution Mix):

For making 10 mL of A.E.S.S.M solution, 1.25 mL of denatured ethanol, 1.0 mL of de-ionized water, and 0.25 mL of IN NaOH (Qualigens) were added to 7.5 mL of Stop solution.

Scintillation Fluid:

For making 2.5 L of scintillating fluid, 1667 mL toluene (Merck), 833 mL triton X-100 (Sigma), 12.5 g 2,5-diphenyloxazole (PPO; Sigma) and 500 mg (1,4-bis(5-phenyl-2-oxazolyl)benzene (POPOP; Sigma) were mixed.

### Working Stock

hDGAT-1 Assay Buffer:

Fresh hDGAT-1 assay buffer containing 0.125% of BSA (free fatty acid, Sigma) was prepared before use.

Substrate Mix Preparation:

Substrate mix was freshly prepared by adding 2047.5 μM of 1,2-dioleoyl-sn-glycerol (19.5 mM; Sigma) and 280 nCi/mL of [¹⁴C]oleoyl-CoA (0.1 mCi American Radiolabeled Chemicals/mL) and the final volume was made up to 1000 μL using hDGAT-1 assay buffer.

hDGAT-1 Enzyme Preparation:

Enzyme was diluted to a working concentration of 1 mg/mL in hDGAT-1 assay buffer, 2.5 μL of the working enzyme stock was used in hDGAT-1 assay (final concentration 25 μg/mL).

### Preparation of Test Samples

The test samples were prepared as follows. A stock solution of 20 mg/mL was prepared for each extract (Extract of Example 1 and Extract of Example 2) in 100% dimethyl sulfoxide (DMSO). The working stock was prepared in hDGAT-1 assay buffer. 10 μL of working stock was added into 100 μL of assay mixture to obtain the final concentration of extracts at 50 μg/mL.

Three different concentrations for dose response (i.e. 25 μg/mL, 50 μg/mL and 100 μg/mL) were prepared for Extract of Example 1 and Extract of Example 2, by serial dilution of stock solution.

Bioactive marker (the compound 1) was tested at 50 μg/mL concentration.

### Assay

60 μL of substrate mix (as described above) was added to a total assay volume of 100 μL. The reaction was started by adding 2.5 μg hDGAT-1 containing microsomal protein and was incubated at 37° C. for 10 min. The reaction was stopped by adding 300 μL of alkaline ethanol stop solution mix (AESSM). The reaction involves the incorporation of radioactive [¹⁴C]oleoyl-CoA into the third hydroxyl group (OH) of 1,2-dioleoyl-sn-glycerol to form the radioactive triglyceride ([¹⁴C]triglyceride) which was then extracted into the upper heptane phase. The radioactive triglyceride product thus formed was separated into the organic phase by adding 600 μL of n-heptane. 250 μL of the upper heptane was added into 4 mL of scintillation fluid and measured using a liquid scintillation counter (Packard; 1600CA) as disintegration per min (dpm) counts. The percentage inhibition was calculated with respect to the vehicle. Results are presented in Table 1.

The dose response was determined at concentrations of 25 μg/mL, 50 μg/mL and 100 μg/mL by serially diluting stock solutions of Extract of Example 1 and Extract of Example 2 in hDGAT-1 assay buffer. Results are presented in Table 2.

TABLE 1
hDGAT-1 inhibition assay

| | | | | % Inhibition of |
|---|---|---|---|---|
| No. | Sample | Concentration | | hDGAT-1 |
| 01 | Extract of Example 1 | 50 | μg/mL | 72.76 |
| 02 | Extract of Example 2 | 50 | μg/mL | 75.94 |
| 03 | The compound 1 | 50 | μg/mL | 85.00 |
| 04 | IN 5530* | 20 | nM | 41.76 |
| 05 | IN 5530* | 0.1 | μM | 71.86 |

*IN 5530: 2-((1s,4s)-4-(4-(4-amino-7,7-dimethyl-7H-pyrimido[4,5-b][1,4]oxazin-6-yl)phenyl)cyclohexyl)acetic acid, which is used a standard, is prepared in-house as per PCT Application Publication No. WO2004/047755 A2

Conclusion: The extracts of the plant Terminalia elliptica (the Extract of Example 1 and the Extract of Example 2) and the bioactive marker (the compound 1) were found to be active in the hDGAT-1 inhibition assay.

TABLE 2
Dose- response in hDGAT-1 inhibition assay

| | | | | % Inhibition of |
|---|---|---|---|---|
| No. | Sample | Concentration | | hDGAT-1 |
| 01 | Extract of Example 1 | 25 | μg/mL | 79.91 |
| 02 | Extract of Example 1 | 50 | μg/mL | 80.62 |
| 03 | Extract of Example 1 | 100 | μg/mL | 83.02 |
| 04 | Extract of Example 2 | 25 | μg/mL | 83.07 |
| 05 | Extract of Example 2 | 50 | μg/mL | 80.55 |
| 06 | Extract of Example 2 | 100 | μg/mL | 83.66 |
| 07 | IN5530* | 20 | nM | 57.87 |
| 08 | IN5530* | 0.1 | μM | 79.94 |

*IN5530: Standard compound, 2-((1s,4s)-4-(4-(4-amino-7,7-dimethyl-7H-pyrimido [4,5-b][1,4]oxazin-6-yl)phenyl)cyclohexyl)acetic acid

Conclusion: Extract of Example 1 and Extract of Example 2 do not show dose dependent in-vitro DGAT-1 inhibition.

### Example 7

### SCD-1 Assay

The assay was carried out according to the method described in reference, European Journal of Pharmacology, 618, 28-36, 2009, the disclosure of which is incorporated by reference for the teaching of the assay.

### Preparation of SCD-1 Enzyme

The SCD-1 enzyme was prepared from rat liver microsomes as described in PCT Publication Application WO2008/074835A1, the disclosure of which is incorporated herein by reference for the teaching of the assay.

Male Sprague-Dawley rats (150-175 g) were fasted for two days and then fed on low fat diet for three days to induce SCD-1 activity. The rats were then sacrificed and their livers were removed and placed on ice. The livers were finely chopped with scissors and then homogenized using a Polytron homogenizer in a homogenization buffer (150 mM KCl, 250 mM sucrose, 50 mM tris-HCl, pH 7.5, 5 mM EDTA, and 1.5 mM reduced glutathione) at 4° C. The homogenate was centrifuged at 1500×g for 20 min at 4° C. The supernatant was collected and centrifuged twice at 10,000×g for 20 min each at 4° C. The resultant supernatant was collected and centrifuged at 100,000×g for 60 min at 4° C. The supernatant was discarded and the microsomal pellet was resuspended in homogenization buffer, aliquoted, and stored at −80° C. The protein content of the resuspended pellet was identified by Bradford assay.

### Preparation of Buffers and Reagents

Preparation of SCD-1 Assay Buffer:

The buffer consisted of 100 mM K₂HPO₄ (Qualigens) and 100 mM NaH₂PO₄.2H₂O (Qualigens), pH 7.4.

Preparation of Potassium Phosphate Buffer:

The buffer consisted of 200 mM K₂HPO₄ (Qualigens), and 200 mM KH₂PO₄ (Qualigens), pH 7.0.

Preparation of SCD-1 Extraction Buffer:

The buffer consisted of 250 mM sucrose (Sigma), 15 mM N-acetyl cysteine (Sigma), 5 mM MgCl₂ (Sigma), 0.1 mM EDTA (Sigma), 0.15 M KCl (Sigma), and potassium phosphate buffer 62 mM, pH 7.0.

Preparation of β-NADH:

A 20 mM stock solution of β-NADH (Sigma) was prepared in SCD-1 assay buffer and stored at −70° C. Working stock of β-NADH was prepared by diluting the stock to 8 Mm with assay buffer just before use.

Preparation of Stearoyl Co-A:

A 1.65 mM stock solution of stearoyl co-A (Sigma) was prepared in SCD-1 assay buffer and stored at −70° C.

Preparation of Radioactive Cocktail:

100 μL of 1 μCi/mL stearoyl (9,10³H) CoA (American Radiolabeled Chemicals) and 144 μL of 1.65 mM stearoyl co-A was added to 5516 μL of SCD-1 assay buffer.

### Preparation of Activated Charcoal Beds in a Multiscreen Plates

A 33% activated charcoal (Sigma) solution was made in assay buffer. 250 μL of the solution was added to each well of a multiscreen plate. The charcoal bed was formed by applying vacuum to the plate through a vacuum manifold. The plates were stored till use.

### Preparation of Test Samples

The test samples were prepared as follows. A stock solution of 20 mg/mL was prepared for each extract (Extract of Example 1 and Extract of Example 2) in 100% dimethyl sulfoxide (DMSO). The working stock was prepared in SCD-1 assay buffer. 10 μL of working stock was added into 100 μL of assay mixture to obtain the final concentration of extracts at 50 μg/mL.

Three different concentrations for dose response (i.e. 25 μg/mL, 50 μg/mL and 100 μg/mL) were prepared for Extract of Example 2 by serial dilution of stock solution.

Bioactive marker (the compound 1) was tested at 50 μg/mL concentration.

### Assay

The microsomes (62.5 μg) were treated with the test sample for 15 min. After which 25 μL β-NADH working stock and 20 μL of radioactive cocktail containing 9,10-³H stearoyl CoA were added and the mixture was incubated at 25° C. for 30 min. The reaction was terminated by the addition of perchloric acid. The plate was then centrifuged and the supernatant from each well was passed through charcoal beds into reservoir plates using the vacuum manifold. The filtrate containing ³H₂O was transferred to scintillation vials containing 4 mL of scintillation fluid and the cpm counts were measured using a liquid scintillation counter. The % inhibition was calculated with reference to the vehicle control.

The dose response was determined at concentrations of 25 μg/mL, 50 μg/mL and 100 μg/mL by serially diluting stock solutions of Extract of Example 2.

A positive control was also assayed with each experiment. Results are presented in Table 3 and Table 4.

TABLE 3
SCD-1 inhibition assay

| No. | Sample | Concentration | | % inhibition of SCD-1 |
|---|---|---|---|---|
| 01 | Extract of Example 1 | 50 | μg/mL | 46.00 |
| 02 | Extract of Example 2 | 50 | μg/mL | 60.65 |
| 03 | The compound 1 | 50 | μg/mL | 74.20 |
| 04 | MF - 152* | 100 | nM | 70.71 |

*MF-152: Standard compound (Bioorganic & Medicinal Chemistry Letters, 19, 5214-5217, 2009).

Conclusion: The Extracts of the plant Terminalia elliptica (Extract of Example 1 and Extract of Example 2) and the bioactive marker (the compound 1) were found to be active in the SCD-1 inhibition assay.

TABLE 4
Dose- response SCD-1 inhibition assay

| | | | |
|---|---|---|---|
| No. | Sample | Concentration | % Inhibition |

| | | | | |
|---|---|---|---|---|
| 01 | Extract of Example 2 | 25 | μg/ml | 44.74 |
| 02 | Extract of Example 2 | 50 | μg/ml | 72.24 |
| 03 | Extract of Example 2 | 100 | μg/ml | 82.17 |
| 04 | MF-152* | 100 | nM | 44.91 |

*MF-152: Standard compound (Bioorganic & Medicinal Chemistry Letters, 19, 5214-5217, 2009).

Conclusion: Extract of Example 2 showed dose related inhibition in the in-vitro SCD-1 inhibition assay.

### Example 8

### Cell Based Triglyceride (TG) Synthesis Assay

Extract of Example 1 and Extract of Example 2 were evaluated for their ability to inhibit triglyceride synthesis in HepG2 cells by the method as reported in reference, European Journal of Pharmacology, 618, 28-36, 2009, the disclosure of which is incorporated by reference for the teaching of the assay.

### Preparation of Buffers, Reagents and Media

Eagle's Minimum Essential Medium (EMEM):

One sachet of powdered EMEM (Sigma) was added to a 1 L conical flask. The empty sachet was rinsed with 10 mL of distilled water. The powder was dissolved in 900 mL distilled water using a magnetic stirrer. 1.5 g sodium bicarbonate (Sigma), 10 mL sodium pyruvate (Sigma) and 1 mL of Penicillin-Streptomycin (Gibco) was also supplemented. After proper mixing the pH was adjusted to 7.2 and the volume made up to 1 L. The medium was filter sterilized and was stored at 4° C.

Inactivated Fetal Bovine Serum (FBS):

Fetal bovine serum (Hyclone) was placed in a water-bath preset at 56° C. for 30 min. The FBS was then aliquoted (45 mL) in 50 mL polypropylene tubes and was stored at −80° C.

Phosphate Buffered Saline (PBS):

Contents of one sachet of PBS (Sigma) were dissolved in 900 mL of distilled water. The pH was adjusted to 7.2 and the volume made up to 1 L. It was then filtered sterilized and was stored at −20° C.

Trypsin-EDTA Solution:

Trypsin-EDTA solution (Sigma) was thawed and aseptically aliquoted (45 mL) in 50 mL polypropylene tubes and was stored at −20° C.

### Preparation of Test Samples

The test samples were prepared as follows. A stock solution of 20 mg/mL was prepared for the Extract of Example 1 and Extract of Example 2, in 100% dimethyl sulfoxide (DMSO). 10 μL of working stock was added into 100 μL of assay mixture to obtain the final concentration of extracts at 50 μg/mL.

Three different concentrations for dose response (i.e. 25 μg/mL, 50 μg/mL and 100 μg/mL) were prepared for Extract of Example 1 and Extract of Example 2 by serial dilution of stock solution.

### Culturing of HepG2 Cells

One frozen vial of HepG2 cells (ATCC No. HB-8065) was thawed in water at 37° C. All the contents of the vial were transferred into a T-75 tissue culture flask containing 9 mL of EMEM and 1 mL inactivated fetal bovine serum. The flask was incubated at 37° C., with 5% CO₂ in a humidity controlled incubator. The flasks were observed for cell growth. When the cells were ˜70% confluent the spent medium was discarded and the cell monolayer was washed with 5 mL of PBS. 1.5-2 mL of Trypsin EDTA solution was added to the flask such that the entire cell layer was covered. When all the cells from the flask were detached, 6 mL of EMEM supplemented with 10% fetal bovine serum was added and mixed to get a uniform cell suspension. The cell suspension was centrifuged at 1000 rpm for 5 min to obtain a pellet of cells. The cell pellet was gently dispersed in 6 mL of EMEM supplemented with 10% fetal bovine serum. Six T-75 flasks were prepared as described above and 1 mL of the cell suspension was added to each of the flasks. The flasks were incubated for 24 h at 37° C. with 5% CO₂ in a humidity controlled incubator. The medium was changed after every 48 h. By 72 h the flasks were ˜70% confluent and ready for plating.

### Assay

A suspension of HepG2 cells was prepared in EMEM medium containing 10% fetal bovine serum. The cell count was determined using a haemocytometer and the count was adjusted to 4×10⁵ cells/mL/well for a 24-well plate. A parallel plate was also made for viability testing to be done at the end of the experiment. The plates were incubated at 37° C. with 5% CO₂ in a humidity controlled incubator till the cells were confluent. When the cells were 70-80% confluent, the medium was discarded and replaced with fresh medium containing the standard compound (MF-152) at 10 μM or Extract of Example 1 or Extract of Example 2 at 50 μg/mL. DMSO was added in vehicle wells at a final concentration of 0.1%. The plates were incubated overnight for ˜18 h. Next day the medium was discarded and replaced with one containing standard compound/extract/DMSO supplemented with 0.1% BSA (fatty acid free).

2 μCi of ¹⁴C labeled acetic acid was also added per well and the plates were further incubated for 6 h at 37° C. after which the medium was discarded and lipids were extracted.

To assess the cytotoxic effects of the plant extracts, the cellular viability test was performed on the parallel plate using MTS (3-(4,5-dimethylthiazol-2-yl)-5-(3-carboxymethoxyphenyl)-2-(4-sulfonyl)-2H-tetrazolium) reagent after 2 h of incubation.

### Lipid Extraction

The extraction was carried out as per the following protocol:

At the end of the experiment, the cells were washed twice with ice-cold PBS. The cells were scrapped into 1 mL cold PBS and pipetted into 15 mL glass tubes containing 4 mL methanol:chloroform (2:1) and was stirred using vortex mixer. The tubes were spun at 4000 rpm for 5 min, and the supernatant was transferred into a new tube. The pellet consisting mostly of proteins was discarded. 1 mL of 50 mM citric acid, 2 mL of water and 1 mL of chloroform was added to the above supernatant and was stirred using vortex mixer. A turbid two phase mixture was obtained. The tubes were spun at 3500 rpm in a non-cooled centrifuge for 15 min. A lower chloroform phase and an upper water/methanol phase were obtained. There was also an inter-phase between the two that consists mostly of precipitated protein. The upper water/methanol phase was discarded, leaving the inter-phase untouched. The lower chloroform phase containing the lipids was transferred into a new tube, and was evaporated on a heating block. The lipids were re-dissolved in 200 μL of chloroform:methanol (2:1). The triglycerides were isolated on TLC silica plates using a solvent system of hexane:diethylether:acetic acid (85:15:0.5). A non-radiolabelled triglyceride standard was run alongside as well as all spots were co-spotted with triglyceride standard. The TLC plates were exposed to iodine vapors and the triglyceride spots were scrapped off and transferred to scintillation vials containing 4 mL of scintillation fluid. The radioactivity was measured in cpm in a liquid scintillation counter and the inhibition was calculated with reference to the vehicle. Results are presented in Table 5.

The dose response was determined at the concentrations of 25 μg/mL, 50 μg/mL and 100 μg/mL by serially diluting stock solutions of Extract of Example 1 and Extract of Example 2. Results are presented in Table 6.

TABLE 5
Inhibition of triglyceride synthesis

| No. | Sample | Concentration | | % Inhibition of TG |
|---|---|---|---|---|
| 01 | Extract of Example 1 | 50 | μg/mL | 80.3 |
| 02 | MF-152* | 10 | μM | 47.46 |
| 03 | Extract of Example 2 | 50 | μg/mL | 69.69 |
| 04 | MF-152* | 10 | μM | 36.5 |

*MF-152: Standard compound (Bioorganic & Medicinal Chemistry Letters, 19, 5214-5217, 2009).

Conclusion: The Extracts of the plant Terminalia elliptica (Extract of Example 1 and Extract of Example 2) were found to be active in the cell based triglyceride synthesis assay.

TABLE 6
Dose response of inhibition of triglyceride synthesis

| | | | | % Inhibition | % |
|---|---|---|---|---|---|
| No. | Sample | Concentration | | of TG | Toxicity |
| 01 | Extract of Example 1 | 25 | μg/mL | 43.70 | 4 |
| 02 | Extract of Example 1 | 50 | μg/mL | 66.70 | 30 |
| 03 | Extract of Example 1 | 100 | μg/mL | 91.07 | 48 |
| 04 | MF-152* | 10 | μM | 22.45 | 7 |
| 05 | Extract of Example 2 | 25 | μg/mL | 32.56 | 0 |
| 06 | Extract of Example 2 | 50 | μg/mL | 63.28 | 2 |
| 07 | Extract of Example2 | 100 | μg/mL | 83.67 | 22 |
| 08 | MF-152* | 10 | μM | 38.25 | 7 |

*MF-152: Standard compound (Bioorganic & Medicinal Chemistry Letters, 19, 5214-5217, 2009).

Conclusion: Extract of Example 1 and Extract of Example 2 showed dose-related inhibition of triglyceride synthesis.

### In Vivo Study

The in vivo experiments were carried out in accordance with the guidelines of the Committee for the Purpose of Control and Supervision of Experiments on Animals (CPCSEA) and with the approval of Institutional Animal Ethics Committee (IAEC).

### Example 9

### Effect of Extract of Example 1 on High Fat Diet (HFD)-Induced Body Weight Gain

The high fat diet (HFD) induced obesity model in rodents has been reported to be a useful model for evaluating the efficacy of anti-obesity agents (Obesity, 17(12), 2127-2133, 2009). It has been reported that feeding a high-fat diet containing 58% kcal fat caused obesity in mice (Metabolism, 47, 1354-1359, 1998). In addition, the mice fed on the high-fat diet has shown significantly higher body weight and significantly heavier visceral adipose tissues (e.g., epididymal, retroperitoneal and mesenteric adipose tissues) than the mice which were fed on the normal diet (Life Sciences, 77, 194-204, 2005).

The HFD induced body weight gain model is reported for evaluating the anti-obesity effects of various natural products (BMC Complementary and Alternative Medicine, 5:9, 1-10, 2005; BMC Complementary and Alternative Medicine, 6:9, 1-9, 2006).

A HFD induced body weight gain study in mice was conducted to evaluate the efficacy of the Extract of Example 1.

Male C57BL/6J mice (in-house; Central Animal Facility, Piramal Healthcare Limited, Goregaon, Mumbai, Maharashtra, India) were acclimatized with HFD (60% Kcal, D12492, Research Diets, USA) for two weeks. Mice exhibiting weight gain were selected for the study and were randomized into treatment groups consisting of 10 mice each.

### Preparation of Test Sample

A suspension of Extract of Example 1 was prepared in polyethyleneglycol 400 (30%) (PEG 400, Fisher Scientific, India) and 0.5% carboxy methylcellulose (70%) (CMC, Sigma, USA).

### Assay

The Extract of Example 1 was administered at a dose of 500 mg/kg body-weight orally, once daily. Orlistat (Biocon, India) was used as the standard drug and was administered orally at a dose of 15 mg/kg body weight, twice daily. A separate group of ten mice was fed a low fat diet (LFD, 10% kcal, D12450B, Research Diet, USA) as a normal control. Vehicle was administered to the HFD and LFD control groups at dose of 10 mL/kg body weight.

The treatments were continued for a period of sixty days. Body weight and feed intake were monitored daily. The % change in body weight (% increase in body weight from day 1) and the cumulative feed intake data was calculated. On day sixtyone, blood samples (˜200 μL/mice) were collected in heparinised (50 IU/mL) micro-centrifuge tubes under isoflurane anesthesia. Plasma was separated by centrifugation at 10000 rpm at 4° C. for estimation of various plasma biochemistry parameters. The biochemistry analysis was performed on BS-400 autoanalyzer (Mindray, China). Subsequently, the mice were sacrificed and following organs/tissues were dissected out and weighed viz., liver, heart, kidneys, epididymal fat and retroperitoneal fat. All the data was analyzed for statistical significance by one-way ANOVA followed by Dunnet's post-hoc test and values of P<0.05 were considered as significant. All analyses were carried out using GraphPad Prism version 4.00 for Windows (GraphPad Software, San Diego, Calif., USA). Results are presented in Table 7, Table 8 and Table 9.

TABLE 7
Effect on HFD induced body weight gain in mice

| | Body weight | Body weight | % Change in |
|---|---|---|---|
| Group | (g, day 0) | (g, day 60) | body weight |
| LFD + vehicle | 24.8 ± 0.7 | 27.1 ± 0.6 | 9.66 ± 1.56** |
| HFD + vehicle | 26.5 ± 0.6 | 32.9 ± 1.0 | 24.28 ± 1.15 |
| HFD + Extract of | 26.7 ± 0.4 | 30.1 ± 0.6 | 14.38 ± 2.36* |
| Example 1 | | | |
| HFD + Orlistat | 26.4 ± 0.4 | 30.9 ± 0.9 | 16.74 ± 2.70* |

*p < 0.05,
**p < 0.01 Vs. HFD + Vehicle; Mean ± S.E.M.

The Extract of Example 1 showed significant inhibition of body weight gain as compared to HFD+ Vehicle group.

TABLE 8
Effect on cumulative feed intake

| | Cumulative feed intake (g/mice) |
|---|---|
| Group | (Day 60) |
| LFD + vehicle | 120.3 ± 3.3 |
| HFD + vehicle | 110.5 ± 2.8 |
| HFD + Extract of | 104.5 ± 4.7 |
| Example 1 | |
| HFD + Orlistat | 119.7 ± 4.7 |

Mean ± S.E.M.

No significant reduction in cumulative feed intake was observed in the Extract of Example 1 when compared to the HFD+ Vehicle group.

TABLE 9
Effect on adipose tissue weight

| | Epididymal | Retroperitoneal | Total |
|---|---|---|---|
| Group | fat (g) | fat (g) | fat ^{#} (g) |
| LFD + vehicle | 0.43 ± .03** | 0.17 ± 0.01** | 0.60 ± 0.05** |
| HFD + vehicle | 1.39 ± 0.13 | 0.65 ± 0.08 | 2.03 ± 0.20 |
| HFD + Extract of | 0.97 ± 0.12* | 0.44 ± 0.05* | 1.41 ± 0.16* |
| Example 1 | | | |
| HFD + Orlistat | 1.07 ± 0.10* | 0.42 ± 0.05* | 1.49 ± 0.15* |

^{#} Total fat = Epididymal fat + Retroperitoneal fat,
*p < 0.05,
**p < 0.01 Vs. HFD + vehicle; Mean ± S.E.M.

Extract of Example 1 showed better reduction in adipose tissue weight in comparison to the HFD+ vehicle group.

The plasma biochemistry analysis for parameters like glucose, triglyceride, cholesterol, alanine aminotransferase, aspartate aminotransferase, albumin, creatinine and urea showed no significant difference between Extract of Example 1 and the vehicle group. The organ weights (heart, liver and kidney) did not show any significant difference.

Conclusion: The treatment of mice on HFD with the Extract of Example 1 caused significant reduction of body weight gain. This reduction in body weight gain was achieved without significant reduction in feed intake and was also evident in the reduced adipose tissue weight (fat mass). Extract of Example 1 has shown antiobesity activity in the high fat diet (HFD) induced obesity model.

### Example 10

### Preparation of Tablet Containing Extract of Terminalia elliptica

| | Ingredients | Function | mg/tab |
|---|---|---|---|
| | Extract of Example 1 | Active ingredient | 500.0 |
| | Microcrystalline cellulose | Diluent | 212.0 |
| | Croscarmellose sodium | Disintegrant | 40.0 |
| | Hydroxypropyl cellulose | Binder | 8.0 |
| | Pregelatinised starch | Disintegrant | 24.0 |
| | Colloidal silicon dioxide | Glidant | 4.0 |
| | Talc | Glidant | 4.0 |
| | Magnesium stearate | Lubricant | 8.0 |
| Core table weight | | | 800.0 |
| | Coating | | |
| | Coating mixture | | 24 |
| | Water | Dispersion medium | |

| | |
|---|---|
| Coated table weight | 824.0 |

### Procedure

Step 1: Weigh 500 mg of the Extract of Example 1 and sieve it through #40 mesh.
Step 2: Weigh 212 mg of Microcrystalline cellulose, 40 mg of Croscarmellose sodium, 8 mg of hydroxylpropyl cellulose and sieve through #40 mesh.
Step 3: Mix the ingredients of step 1 with the ingredients of step 2 in a non shear blender for 10 min.
Step 4: Compact the blend using appropriate compactor.
Step 5: Mill the flakes obtained using suitable size screen to obtain the desired particle size. Repeat the process till the desired amount of granules are obtained.
Step 6: Weigh extragranular excipients namely Pregelatinised starch, colloidal silicon dioxide, Talc and sieve the ingredients through #40 mesh.
Step 7: Mix the ingredients of step 6 with the granules of step 5 for 15 min in non shear blender.
Step 8: Weigh 8 mg of magnesium stearate and sieve it through #60 mesh.
Step 9: Mix the sifted magnesium stearate with step 7 blend for 2 min.
Step 10: Compress the blend with a desired tooling.

### Preparation of Coating Solution

Step 1: Disperse the coating material in required quantity of water.

### Step 2: Homogenize for 30 min.

Step 3: Filter the solution through nylon cloth.
Step 4: Coat the tablets to get a desired weight gain.
Step 5: Dry the tablets in the coating pan for about 20-30 min.

1-13. (canceled)

14. A composition comprising a therapeutically effective amount of standardized extract of the plant Terminalia elliptica as an active ingredient along with at least one pharmaceutically acceptable carrier; wherein the extract contains ellagic acid, 4-O-alpha-L-rhamnopyranoside as the bioactive marker.

15. The composition as claimed in claim 14, wherein the said composition contains 5%-100% of the extract of the plant Terminalia elliptica.

16. The composition as claimed in claim 14, wherein the extract is obtained from the bark of the plant Terminalia elliptica.

17. The composition as claimed in claim 14, wherein the extract of the plant Terminalia elliptica contains 0.01% to 10.0% of the compound 1, as the bioactive marker.

18. The composition as claimed in claim 14, wherein the said composition is administered orally.

19. The composition as claimed in claim 18, wherein the composition is formulated for oral administration in the form of a tablet, capsule or granules.

20. A method for the treatment of a metabolic disorder comprising administering to a subject in need thereof a therapeutically effective amount of the composition as claimed in claim 14.

21. The method as claimed in claim 20, wherein the metabolic disorder is selected from insulin resistance, hyperglycemia, diabetes mellitus, obesity, glucose intolerance, hypercholesterolemia, dyslipidemia, hyperinsulinemia, atherosclerotic disease, polycystic ovary syndrome, coronary artery disease, metabolic syndrome, hypertension, a disorder associated with abnormal plasma lipoprotein, triglycerides or a disorder related to pancreatic beta cell regeneration.

22. The method as claimed in claim 21, wherein the metabolic disorder is insulin resistance, diabetes mellitus, hyperglycemia, metabolic syndrome, glucose intolerance, obesity, dyslipidemia, a disorder associated with abnormal plasma lipoprotein, triglycerides or a disorder related to pancreatic beta cell regeneration.
